(19) United States
(12) Patent Application Publication
(10) Pub. No.: US 2021/0140961 A1
(43) Pub. Date: May 13, 2021

(54) Methods of Preserving Blood Samples for Mass Screening to Detect at-Risk Individuals for Autoimmune Diseases

(71) Applicant: TheraTest Labs Inc., Lombard, IL (US)

(72) Inventors: Mihai Teodorescu, Lombard, IL (US); Sunil S. Metkar, Lombard, IL (US); Marius Teodorescu, Lombard, IL (US)

(21) Appl. No.: 17/092,999

(22) Filed: Nov. 9, 2020

Related U.S. Application Data
(60) Provisional application No. 62/932,866, filed on Nov. 8, 2019.

(51) Int. Cl. G01N 33/564; A01N 1/02

(52) U.S. Cl. CPC G01N 33/564; G01N 2800/52; G01N 2800/102; A01N 1/021

(57) ABSTRACT

A method of screening an individual for an autoimmune disease comprising analyzing a blood sample, containing at least one biomarker, from an individual wherein the blood sample is suspended and preserved by a blood stabilizing solution and wherein the autoimmune disease is rheumatoid arthritis, celiac disease, systemic lupus erythematosus, or Sjogren's syndrome.

### BACKGROUND

This application claims priority to U.S. Provisional Application No. 62/932,866 filed on Nov. 8, 2019.

Rheumatoid arthritis is a chronic debilitating and crippling inflammatory disease that affects about 1.3 million individuals in this country or about 1% of the adult population, mostly women. Each year about 40 new patients for every 100,000 people are diagnosed with rheumatoid arthritis and that number is increasing. Diagnosis of the disease for most people, and particularly those of the low socio-economic status, is relatively late. During the past 30 years, significant progress has been made in the management of rheumatoid arthritis with two classes of drugs: synthetic disease-modifying anti-rheumatic drugs (DMARDs) and biologics. The former are relatively inexpensive, but with limited benefits for most patients under the current diagnostic timeframe. Biologics are more effective, but substantially more expensive. The 2018 annual direct costs of treatment for the 1.3 million rheumatoid arthritis patients in the United States was estimated at $16.2 billion.

The scientific community has accumulated convincing data about the need to diagnose the disease as early as possible in order to treat the disease during the “window of opportunity” with the goal of achieving long term disease-free and possibly treatment-free remission. The “window of opportunity” is defined as an early time in the disease process, when treatment may be relatively inexpensive and also effective, in changing the long-term outcome. Not surprisingly, cost reduction has multiple government and private stakeholders to include Medicare, Medicaid, insurance companies, state treasury departments etc. By using the methods disclosed herein for diagnosis and subsequent treatment, it is estimated that the early discovery and diagnosis of the disease through mass screening will cost less than $2 billion/year.

As disclosed herein, scientific studies have shown that the two biomarkers or risk factors of rheumatoid arthritis autoimmunity—rheumatoid factors (RF) and anti-cyclic citrullinated peptide antibodies (ACPA)—are present in the blood serum of patients many years before the development of symptoms. Unfortunately, these risk factors are typically discovered after the treatment “window of opportunity.” The disclosed methods of screening herein include two unique advantages that offer the opportunity for early low cost discovery of rheumatoid arthritis by the mass screening: 1) very high (at least 88%) sensitivity as well as high specificity (at least 95-99%) and 2) the methods can be performed with only one drop of whole blood from a finger prick.

The present disclosure provides methods to identify individuals who have, serologically, the highest risk of developing rheumatoid arthritis by measuring rheumatoid arthritis disease biomarkers in whole blood that is collected by finger prick and suspended and preserved by using a blood stabilizing solution (“Therazyme™”). The solution preserves the blood samples and the associated biomarkers/antibodies used to identify individuals with an increased risk of developing rheumatoid arthritis.

### SUMMARY

This Summary is provided to introduce a selection of concepts in a simplified form that are further described below in the Detailed Description. The Summary is not intended to identify key features or essential features of the claimed subject matter, nor is it intended to be used to limit the scope of the claimed subject matter.

According to one aspect, a method of stabilizing a blood sample for serologic analysis is disclosed. In one example, the method may include obtaining a blood sample in which the blood sample includes at least one biomarker, and suspending the blood sample in a blood stabilizing solution. In certain examples, the amount of the blood stabilizing solution may be sufficient to preserve the biomarker. In other examples, the blood stabilizing solution may include tris-HCl in buffered saline, bovine serum albumin, tyrosine, calcium chloride, trehalose, a preservative, and water. In some examples, a concentration of the bovine serum albumin may be at least 0.5%, a concentration of the tyrosine may be at least 0.04%, a concentration of the calcium chloride may be at least 0.05%, and a concentration of the trehalose may be at least 1.0%.

In other examples, the method of stabilizing a blood sample for serologic analysis may include a concentration of the tris-HCl in buffered saline of at least 0.1M. In some examples a concentration of the preservative may be at least 0.001%. In yet other examples, the preservative may be 2-methyl-4-isothiazolin-3-one solution. In certain examples the biomarker may be preserved for at least 7 days. In other examples, the biomarker may identify an autoimmune disease. In still other examples, the biomarker may be an immunoglobulin such as rheumatoid factor IgM, rheumatoid factor IgA, anti-cyclic citrullinated peptide, or combinations thereof.

In other examples, the method of stabilizing a blood sample for serologic analysis may be used to screen an individual for an autoimmune disease by analyzing a blood sample from an individual. In some examples, the autoimmune disease is rheumatoid arthritis, celiac disease, systemic lupus erythematosus, or Sjogren's syndrome.

According to another aspect, a method of mass screening individuals for rheumatoid arthritis is disclosed that may include screening a general population, identifying an individual with a high risk factor for developing rheumatoid arthritis, collecting a blood sample from the individual wherein the blood sample includes at least one biomarker, and suspending the blood sample in a stabilizing solution. In some examples the stabilizing solution may include an amount of the solution is sufficient to preserve the biomarker. In other examples, the solution may include tris buffered saline, bovine serum albumin, tyrosine, calcium chloride, trehalose, a preservative, and water. In other examples the concentration of the bovine serum albumin may be at least 0.5%, the concentration of the tyrosine may be at least 0.04%, the concentration of the calcium chloride may be least 0.05%, and the concentration of the trehalose may be at least 1.0%. In still other examples, the method may include analyzing the blood sample to determine a biomarker type and a biomarker level, and determining if the individual is at high risk for developing rheumatoid arthritis based on the biomarker type and the biomarker level.

In other examples, the blood sample may be collected via a finger prick and the blood sample may be at least 20 μL of whole blood. In some examples, the biomarker may be rheumatoid factor IgM, rheumatoid factor IgA, or an anti-cyclic citrullinated peptide. In yet other examples, the concentration of the tris buffered saline may be about 0.1 M and the concentration of the preservative solution may be at least 0.001%. In another example, the biomarker is preserved for at least 7 days. In still another example, the biomarker type and the biomarker level may be determined by an ELISA constructed to identify rheumatoid factor IgM, rheumatoid factor IgA, and anti-cyclic citrullinated peptide.

In one example, a biomarker level of rheumatoid factor IgM and rheumatoid factor IgA and anti-cyclic citrullinated peptide higher than about 95% of a normal population may indicate a high risk of developing rheumatoid arthritis. In another example, a biomarker level of anti-cyclic citrullinated peptide higher than about 95% of a normal population may indicate a high risk of developing rheumatoid arthritis.

According to another aspect, a kit for mass rheumatoid arthritis screening is disclosed. The kit may include a device to obtain a blood sample from an individual, and a blood sample collection vial including a label and a blood stabilizing solution. In some examples, the stabilizing solution may include an amount of the solution is sufficient to preserve a biomarker. In other examples, the solution may include tris buffered saline, bovine serum albumin, tyrosine, calcium chloride, trehalose, a preservative, and water. In other examples the concentration of the bovine serum albumin may be at least 0.5%, the concentration of the tyrosine may be at least 0.04%, the concentration of the calcium chloride may be least 0.05%, and the concentration of the trehalose may be at least 1.0%. In still other examples, the biomarker may be rheumatoid factor IgM, IgA, or an anti-cyclic citrullinated peptide.

According to another aspect, kit for screening an individual for an autoimmune disease is disclosed. The kit may include a device to obtain a blood sample from an individual, and a blood sample collection vial including a label and a blood stabilizing solution. In some examples, the stabilizing solution may include an amount of the solution is sufficient to preserve a biomarker. In other examples, the solution may include tris buffered saline, bovine serum albumin, tyrosine, calcium chloride, trehalose, a preservative, and water. In other examples the concentration of the bovine serum albumin may be at least 0.5%, the concentration of the tyrosine may be at least 0.04%, the concentration of the calcium chloride may be least 0.05%, and the concentration of the trehalose may be at least 1.0%.

In some examples, the biomarker may identify an autoimmune disease. In still other examples, the autoimmune disease may be rheumatoid arthritis, celiac disease, systemic lupus erythematosus, or Sjogren's syndrome.

According to another aspect, a non-transitory machine-readable medium storing instructions is disclosed that, when executed by one or more processors, may cause the one or more processors to perform steps including screening a general population, identifying an individual with a high risk factor for developing rheumatoid arthritis, analyzing a blood sample collected from the individual in which the blood sample includes at least one biomarker to determine a biomarker type and a biomarker level, and determining if the individual is at high risk for developing rheumatoid arthritis based on the biomarker type and the biomarker level.

This Summary is provided to introduce a selection of concepts in a simplified form that are further described below in the Detailed Description. The Summary is not intended to identify key features or essential features of the claimed subject matter, nor is it intended to be used to limit the scope of the claimed subject matter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present invention is illustrated by way of example and not limited in the accompanying figures in which like reference numerals indicate similar elements and in which:

FIG. 1 is a graph showing the rheumatoid arthritis biomarker levels for IgM and IgA in diluted whole blood samples suspended and preserved by the methods according to one or more aspects described herein; and

FIG. 2 is a graph showing the rheumatoid arthritis biomarker levels for cyclic citrullinated peptide 2 (cCP2) in diluted whole blood samples suspended and preserved by the methods according to one or more aspects described herein.

Further, it is to be understood that the drawings may represent the scale of different components of one single embodiment; however, the disclosed embodiments are not limited to that particular scale.

### DETAILED DESCRIPTION

In general, aspects of this disclosure relate to methods of preserving blood samples for the screening of autoimmune diseases, and more specifically, methods of mass screening for rheumatoid arthritis.

Rheumatoid arthritis is a chronic autoimmune inflammatory disease with a lifetime risk of 3.6% and 1.7% for women and men, respectively. At this time about 1% of the adult US population has risk of developing rheumatoid arthritis, or 1.3 million, with about 41 new cases per 100,000 individuals identified each year. And with a frequency that is increasing (Myasoedova, Davis et al. 2010) (Myasoedova, Crowson et al. 2010). It is mostly an insidious disease with progressive arthralgia and other systemic manifestations. It may start in one or a few joints, mostly in the upper limbs and progresses to multiple joints, symmetrically, hands, elbows, shoulders, knees, and also the neck. The inflammation is located mainly in the synovium which becomes thick, palpable, and the inflammatory process leads to bone erosions (Brasington 2019). In addition, patients manifest prolonged morning stiffness, fatigue, lung disease independent of smoking (Sparks, Chang et al. 2016), anemia etc. Left untreated or when treatment is ineffective, progressive joint destruction leads to deformities, loss of mobility and general disability and those affected become incapacitated and depressed. And mortality is significantly increased (Symmons, Jones et al. 1998, Gabriel, Crowson et al. 1999, Kvalvik, Jones et al. 2000, Tomasson, Aspelund et al. 2010, Sparks, Chang et al. 2015).

Currently, there are three main categories of drugs: a) symptomatic treatment with non-steroidal anti-inflammatory drugs, such as aspirin and ibuprofen + steroids which have limited benefits and significant side effects; b) relatively inexpensive synthetic disease-modifying anti-rheumatic drugs (DMARDs) (Saunders, Capell et al. 2008) such as methotrexate, sulfasalazine, hydroxychloroquine, plus steroids (e.g. prednisone) and symptomatic pain control; and c) biological DMARDS, i.e. monoclonal antibodies targeting cells or critical molecules (e.g. Infliximab, Tocilizumab, Rituximab, Golimumab, Certolizumab pegol, Adalimumab; and also recently developed small molecules targeting specific intra-cellular mechanisms of the inflammatory process (e.g. Tofacitinib, Abatacept, Etanercept). DMARDs are effective in modifying the course of the disease and can potentially lead, in a minority of patients, to the state of “relative cure” (i.e. a state of remission of the disease that no longer requires treatment—drug-free remission) (Baker, Skelton et al. 2019). They are generally affordable, but their efficacy varies from one patient to another due to multiple factors. One of the main factors is the stage of the disease when the treatment started. They are most effective during the “window of opportunity” (O′Dell, Curtis et al. 2013), even when we consider variable patient response (Willemze, van der Linden et al. 2011, Contreras-Yanez and Pascual-Ramos 2015, Nagy and Van Vollenhoven 2015, Burgers, Raza et al. 2019). There is evidence suggesting that early treatment during the “window of opportunity” with DMARDs may lead to a state of prolonged disease-free or even drug-free remission (Baker, Skelton et al. 2019). Finally, there is a new generation of small synthetic molecules that target the inflammatory process, but are relatively more expensive. They have shown significant benefits in terms of efficacy and chance of achieving drug-free remission and are generally used after the failure of synthetic DMARD treatment (Hresko, Lin et al. 2018).

Since delay in effective treatment beyond the “window of opportunity” is also a major cause of long term escalation of costs, the methods of quantitative and qualitative mass screening disclosed herein provide a solution to such costs. For the quantitative screening, detection of biomarkers including autoantibodies and/or of isotypes of autoantibodies in the blood, or a combination thereof, are useful in identifying an individual at risk of developing rheumatoid arthritis.

To identify the individuals who have the highest risk of developing rheumatoid arthritis in the future based on serological markers, a blood sample is analyzed using a high throughput and largely automated enzyme-linked immunosorbent assay (ELISA) protocol. An ELISA assay, well-known by one of skill in the art, uses a solid-phase enzyme immunoassay to detect the presence of a protein, for example, in a liquid sample using antibodies. Antigens from the sample, for example, are attached to a surface, a matching antibody is then applied over the surface so it can bind to the antigen. The antibody is linked to an enzyme, and in the final step, a substance containing the enzyme's substrate is added. The subsequent reaction produces a detectable signal, most commonly a color change. Performing an ELISA involves at least one antibody with specificity for a particular antigen (i.e., a biomarker). Antibodies are also known as an immunoglobulins (Ig) and may be referred to as biomarkers herein. Generally, there are two major types of biomarkers to include biomarkers of exposure, which are used in risk prediction, and biomarkers of disease, which are used in screening and diagnosis and monitoring of disease progression. Human antibodies are classified into five isotypes (IgM, IgD, IgG, IgA, and IgE). The sample with an unknown amount of antigen is immobilized on a solid support. After the antigen is immobilized, the detection antibody is added, forming a complex with the antigen. The detection antibody can be covalently linked to an enzyme or can itself be detected by a secondary antibody that is linked to an enzyme through bioconjugation. Between each step, the plate is typically washed with a mild detergent solution to remove any proteins or antibodies that are non-specifically bound. After the final wash step, the plate is developed by adding an enzymatic substrate to produce a visible signal, which indicates the quantity of antigen in the sample.

In addition to detecting biomarkers or genetic markers for rheumatoid arthritis, the methods disclosed herein may be used to detect and identify biomarkers or genetic markers related to other autoimmune diseases. Other autoimmune diseases like Crohn's disease, ulcerative colitis, psoriasis and systemic lupus erythematosus are chronic, debilitating disorders in which the body mounts an abnormal immune response against its own organs and tissues. In some examples, the biomarkers may include amphiregulin, B cell-activating factor, cartilage oligomeric matrix protein, CD163, collagen IV, complement C3, complement Factor H - related protein 1, ficolin-3, haptoglobin, interferon gamma, interferon gamma induced protein 10, interleukin-1 alpha, interleukin-1 beta, interleukin-10, interleukin-12 subunit p40, interleukin-12 subunit p′70, interleukin-17, interleukin-23, interleukin-6, interleukin-6 receptor, interleukin-6 receptor subunit beta, macrophage Inflammatory protein-3 alpha, macrophage migration inhibitory factor, matrix metalloproteinase-10, matrix metalloproteinase-3, monocyte chemotactic protein 1, monocyte chemotactic protein 3, osteopontin, thrombomodulin, thyroglobulin, thyroglobulin antibody, tumor necrosis factor alpha, vascular cell adhesion molecule-1, or combinations thereof.

In accordance with the methods disclosed herein, by using whole blood collected from an individual and preserving the blood in the stabilizing diluent, the blood sample can subsequently be analyzed via ELISA to identify individuals who are at risk of developing rheumatoid arthritis or other autoimmune disease based upon the type and amount of biomarkers in the blood sample. The type and amount of biomarkers in the blood sample may also be processed with an individual's prior screening data to determine ultimate risk.

The methods disclosed herein use an ELISA specifically constructed to identify IgM and IgA RF as well as ACPA. Notably, IgG RF alone is extremely rare in rheumatoid arthritis. The red cells from a blood sample are allowed to settle in the stabilizer solution disclosed herein. A multichannel pipette is used to transfer 100 μL aliquots from the samples are transferred to a 96-well plate coated with rabbit IgG and 100 μL are transferred to a plate coated with cyclic citrullinated peptide to measure ACPA. The loaded plates are then processed and analyzed for the presence of biomarkers. As disclosed herein, the identification of specific risk biomarkers (i.e., type and amount) are analyzed in view of an individual risk evaluation. According to the methods disclosed herein, the risk evaluation may include factors such as gender, age, race, joint pain history, alcohol consumption, smoking, body weight/height, stress, diet, periodontal disease, etc.

It is well-known in the art that autoantibodies appear well before the onset of any autoimmune disease, including rheumatoid arthritis (Leslie, Lipsky et al. 2001, Emery, Mankia et al. 2017). Their presence is a major risk factor for future rheumatoid arthritis development (Aletaha, Neogi et al. 2010). Rheumatoid arthritis and other autoimmune diseases have an unusual shift in the physiologic sequence of autoantibodies and/or isotype production. In most cases, the first autoantibodies produced are of the IgM isotype. With T-cell assistance, B cells can switch production from RF IgM to IgA and/or IgG. The actual findings of RF isotypes in long-term serological studies of IgA RF, IgM RF, or both IgM and IgA, were identified (del Puente, Knowler et al. 1988, Jonsson, Thorsteinsson et al. 1992, Jonsson, Arinbjarnarson et al. 1995, Jonsson and Valdimarsson 1998, Rantapaa-Dahlqvist, de Jong et al. 2003, Gan, Trouw et al. 2015, Brink, Hansson et al. 2016, Kelmenson, Wagner et al. 2019). The abnormal production of antibodies may be explained if the RF was derived from cross-reactivity with antibodies against other antigens from pathogenic infectious agents, including intestinal or gingival bacteria (Horta-Baas, Romero-Figueroa et al. 2017, Tracy, Buckley et al. 2017). As a result, the antibodies may have already been highly mutated and isotype-switched. Changes in RF and ACPA features may occur before the development of clinical rheumatoid arthritis indicating that additional evolution of the antibody response may be necessary for pathogenesis. This includes an increase in antibody levels shortly before onset of rheumatoid arthritis (del Puente, Knowler et al. 1988) and/or binding strength (affinity), epitope spreading, or changes in glycosylation profile (Falkenburg and van Schaardenburg 2017). The ELISA protocol used in the methods disclosed herein takes advantage of the presence of the combination between IgA and IgM RF and as well as IgG RF, and their corresponding levels. A prior study found that, among 9712 individuals without rheumatoid arthritis, 183 subsequently developed rheumatoid arthritis (Nielsen, Bojesen et al. 2012). The 10 year risk of developing rheumatoid arthritis was 3.6 times higher than normal for low RF levels and 26 times for those with high levels of RF.

Further, the presence of multiple isotypes of RF may define increased rheumatoid arthritis risk. Prior studies have indicated that individuals with elevated IgA RF combined with either IgM or IgG were at higher risk for developing rheumatoid arthritis and IgA RF was the best predictor of bone erosions (Jonsson, Thorsteinsson et al. 1992). However, the studies also indicated that measurement of IgM isotype only did not contribute significantly to predicting increased risk of developing rheumatoid arthritis (Houssien, Jonsson et al. 1998).

Anti-citrullinated protein antibodies bind to proteins in which arginine amino acid residues have been enzymatically converted into citrulline (Schellekens, de Jong et al. 1998). The most common ACPA is anti-CCP/2 and is included in the methods described herein for screening individuals for a risk of developing rheumatoid arthritis. In addition, chronic inflammation of infectious or non-infectious origin in the gums, intestines, or lungs (smoking, silica) may initiate an enzymatic process that creates a neo-epitope (Kim, Jiang et al. 2015, Horta-Baas, Romero-Figueroa et al. 2017, Joshua, Chatzidionisyou et al. 2017, Zaccardelli, Friedlander et al. 2019). This neo-epitope fits in the predisposed “pocket” HLA-DRβ31 of genetically predisposed individuals. The antigen presentation to T-cells leads to induction of IgG, IgA, and ACPA (Kim, Jiang et al. 2015, Deane, Demoruelle et al. 2017, Hedström, Ronnelid et al. 2019, Okada, Eyre et al. 2019). This proposed mechanism explains the increased of developing rheumatoid arthritis represented by inflammation in the lungs induced by smoking and other factors in genetically predisposed individuals (Sparks and Karlson 2016). Indeed, smoking cessation was shown to reduce the risk for rheumatoid arthritis in at-risk populations (Liu, Tedeschi et al. 2019). As such, periodontal disease (i.e., gingival related problems) and inflamed intestines may also explain the generation of ACPA and/or IgA RF in the pathogenesis of rheumatoid arthritis (Cheng, Meade et al. 2017, Deane, Demoruelle et al. 2017). Certain periodontal bacteria, including Porphyromonas gingivalis and Aggregatibacter actinomycetemcomitans may contribute to autoantibody production in rheumatoid arthritis through direct post-translational modification of proteins or, indirectly, by influencing neutrophil-mediated neo-epitope generation (citrullination). Oral bacteria, like Porphyromonas gingivalis that invade the blood may also contribute to chronic inflammatory responses and generation of autoantibodies. Anaeroglobus and Prevotella species have been found in fecal samples of patients with early rheumatoid arthritis. Prior studies have shown evidence of differences in the microorganisms at oral and gastrointestinal mucosal sites between patients with early rheumatoid arthritis and patients with established treated disease and healthy controls (Cheng, Meade et al. 2017, Deane, Demoruelle et al. 2017).

Testing for ACPA has become one of the criteria for the routine laboratory diagnosis of rheumatoid arthritis (Nishimura, Sugiyama et al. 2007). The spectrum of serologic biomarkers that can be combined to identify individuals at risk for developing rheumatoid arthritis is constantly expanding. Recently, antibodies against carbamylated proteins (Shi, van de Stadt et al. 2014, Gan, Trouw et al. 2015) (anti-CarP Abs), which bind to proteins in which lysine has been chemically converted into homocitrullines, have also been described in rheumatoid arthritis. Rheumatoid factors and ACPAs, as well as anti-CarP Abs, can be found in serum samples taken years before the onset of clinical rheumatoid arthritis (Aho, Heliovaara et al. 1991, Rantapaa-Dahlqvist, de Jong et al. 2003, Nielen, van

Schaardenburg et al. 2004, Majka, Deane et al. 2008, Chibnik, Mandl et al. 2009, Shi, van de Stadt et al. 2014, Gan, Trouw et al. 2015, Kelmenson, Wagner et al. 2019). Autoantibodies against other novel peptides have been identified in rheumatoid arthritis (De Winter, Hansen et al. 2016, Falkenburg and van Schaardenburg 2017) and were also detected in a small proportion of ACPA-negative patients. For rheumatoid arthritis patients, the MUCSB gene and anti-MAA autoantibodies (malondialdehyde-acetaaldehyde) acetaaldehyde) are risk factors for development of chronic obstructive pulmonary disease (Thiele, Duryee et al. 2015, Juge, Lee et al. 2018) independent of smoking (Sparks, Chang et al. 2016). Future studies may also identify new autoantibody markers or characteristics of individuals at risk of developing rheumatoid arthritis (Young, Deane et al. 2013, Mankia and Emery 2016, Emery, Mankia et al. 2017, Falkenburg and van Schaardenburg 2017). These studies may be helpful to further identify patients at higher risk for rheumatoid arthritis. Additionally, increased levels of ACPA anti-CarP, peptidyl arginine deiminase type 4 (PAD4) and cytokines appear prior to rheumatoid arthritis onset (Sokolove, Bromberg et al. 2012, Gan, Trouw et al. 2015) (Kolfenbach, Deane et al. 2010).

Biomarker screening methods disclosed herein include a rabbit IgG that is the antigen in the high sensitivity and high specificity RF/3 isotype testing (Swedler, Wallman et al. 1997) (Table 3). The use of rabbit antigen explains the well-known high specificity of the Waller-Rose test compared to latex agglutination or nephelometry, where the antigen is human IgG. A meta-analysis (Nishimura, Sugiyama et al. 2007) provides a comparative background for the favorable performance characteristics of the test disclosed herein (test data included in the meta-analysis (Swedler, Wallman et al. 1997)). Further, the combined presence of RF and ACPA can achieve a positive predictive value for a combined presence of RF and ACPA can achieve positive predictive value for rheumatoid arthritis close to 100% (Raza, Breese et al. 2005). Notably, the RF directed against rabbit IgG is relatively specific for rheumatoid arthritis. This observation led to the development of the Rose-Waaler agglutination method for the diagnosis of rheumatoid arthritis, where the antigen is rabbit IgG on the surface of sheep red cells (del Puente, Knowler et al. 1988, Del Puente, Knowler et al. 1989). The Rose-Waaler test was replaced by ELISA including rabbit IgG as the antigen. It is just as specific for rheumatoid arthritis, but much more sensitive, amenable to automation, and allows for the measurement of all RF isotypes (Jonsson,

Thorsteinsson et al. 1992). Routine clinical nephelometry, however, measures only of RF IgM against human IgG, which is less sensitive and specific compared to using rabbit IgG. Information collected on IgG RF, however, should be interpreted with caution. Without pepsin digestion, IgG-RF measurements are susceptible to false positives due to Fc-Fc interaction. This phenomenon can occur when IgG4 antibodies in serum bind with their Fc domain to the Fc domain of the IgG used as target antigen in IgG-RF assays (Jonsson, Thorsteinsson et al. 1995, Zack, Stempniak et al. 1995, Jonsson, Thorsteinsson et al. 2000, Rispens, Ooievaar-De Heer et al. 2009). The IgG RF identified by the methods disclosed herein is specific since it measures only the binding of F(ab)′2 fragments of IgG after pepsin digestion, and IgM is destroyed and the contribution of IgA is negligible (Swedler, Wallman et al. 1997). The poor specificity of IgG RF results is reflected in a major difference: IgG alone is almost non-existent in rheumatoid arthritis patients when only the F(ab)′2 fragments are detected after pepsin digestion (Swedler, Wallman et al. 1997), but is present in studies with no digestions (Kelmenson, Wagner et al. 2019). In the methods disclosed herein, IgG RF, when present together with IgM and IgA, results in a specificity of RF is about 99% and the positive predictive value is about 96% (Swedler, Wallman et al. 1997). Only in hepatitis C infection and Sjogren's syndrome can mimic rheumatoid arthritis with all three biomarkers. Table 1 below shows the distribution of RF biomarkers and anti-CCP/2 biomarkers in a sample of clinically diagnosed rheumatoid arthritis patients according to the methods described herein.

TABLE 1

| RF Isotypes | 88.35% positive |
|---|---|
| IgM + IgG + IgA | 44.29% |
| IgM + IgA only | 33.52% |
| IgM only | 6.52% |
| IgA only | 3.67% |
| IgG only | 0.32% |
| IgM + IgG only | 0.08% |
| IgG + IgA only | 0.03% |
| RF negative | 11.65% |
| RF negative CCP2 positive | 2.3% |
| CCP2 positive | 80.10% |

Isolated RF, particularly IgM, may be found in normal individuals, in various infections

(Newkirk, Goldbach-Mansky et al. 2005) and in first degree relatives of patients with RA (Ioan-Facsinay, Willemze et al. 2008). Having more than one biomarker for rheumatoid arthritis (i.e., IgM+IgA or IgM+IgG+IgA) is very rare in random healthy blood bank donors (Swedler, Wallman et al. 1997). The diagnostic significance of an isolated increase in IgA RF is still not fully understood, since it is also found frequently in patients with various connective tissue diseases, and it may suggest chronic inflammation (Jonsson, Thorsteinsson et al. 1992, Jonsson, Arinbjarnarson et al. 1995, Jonsson, Thorsteinsson et al. 1995, Swedler, Wallman et al. 1997, Jonsson and Valdimarsson 1998).

The methods disclosed herein primarily rely upon both RF isotypes and ACPA biomarkers to identify individuals at risk for rheumatoid arthritis. Indeed, having at least IgM and IgA isotypes correlates to an increased risk of developing rheumatoid arthritis, whereas having only one isotype generally does not (Jonsson, Thorsteinsson et al. 2000). In addition, having both RF and ACPA biomarkers has a discovery specificity for early rheumatoid arthritis of 65-100% with a sensitivity of 59-88%, according to a meta-analysis (Verheul, Bohringer et al. 2018). Additionly, anti-carbamylated Ab further increases the specificity, but with a significant loss of sensitivity (Swedler, Wallman et al. 1997).

The routine clinical RF testing by nephelometry (turbidimetry) is inappropriate for two main reasons: 1) it measures mostly IgM RF and requires venous blood to provide sufficient serum. In addition to RF and ACPA levels, information from the personal evaluator (See Appendix), including status as first degree relative of patients with RA or SLE (Sparks, Iversen et al. 2014, James, Chen et al. 2019) will be used in the selection algorithm (Fig . . . ). For example, any seropositive individual associated with arthralgia and family history or RA and/or SLE may increase by at least 4 fold the chance of developing RA (Sparks, Chen et al. 2014, Jiang, Frisell et al. 2015) (Van De Stadt, Witte et al. 2013). In the future the algorithm may be expanded to include the DRB1 status, in light of the value of the shared epitope in the development of RA (Sparks, Chang et al. 2016). ACPA+RF IgM+arthralgia patients have a higher risk of developing RA than single positive (Bos, Wolbink et al. 2010), and also have more and larger bone erosions and enhanced bone marrow edema on Mill (Boeters, Nieuwenhuis et al. 2016).

Furthermore, double positivity, but not single positivity, is associated with higher disease activity than ACPA-positive RF-negative patients. They also have higher CRP and pro-inflammatory cytokine profiles than single-positive patients (Sokolove, Johnson et al. 2014).

By identifying individuals with an increased risk for rheumatoid arthritis according to the methods disclosed herein, preventing rheumatoid arthritis may include a first or primary method that seeks to prevent the disease from developing (Majka and Holers 2003). A secondary method that addresses treatment of the disease state, considering that spontaneous recovery may also occur (Bos, Wolbink et al. 2010). And a tertiary method that aims to return the individual with established disease to a healthy state by treatment and rehabilitation. Primary prevention for rheumatoid arthritis could be the discovery of unmodifiable risk factors, such as combinations of autoantibodies and/or a genetic link or genetic biomarker to a relative with rheumatoid arthritis, or other systemic autoimmune disease (Sparks, Iversen et al. 2014, Sparks, Iversen et al. 2018).

Secondary prevention of rheumatoid arthritis is also dependent on the discovery of the same unmodifiable risk factors and also on obtaining evidence that the early rheumatoid arthritis is likely to progress. Both objective and subjective findings are required. The presence of autoantibodies, arthralgia, ultrasound, MRI and cytokine changes before the disease fully develops are valuable elements. MRI of the symptomatic joints of the hands and feet of ACPA-positive individuals without clinical arthritis have revealed evidence of bone marrow edema of wrist, MCP, PIP and MTP joints in some, but not all patients (Krabben, Stomp et al. 2013). The correlation with pain and serological data is critical. MRI-detected synovitis, bone marrow edema and tenosynovitis were all shown to be associated with future arthritis development (van Nies, Alves et al. 2015, van Steenbergen, Mangnus et al. 2016).

Ultrasound has also been used as an imaging modality to assess the presence of synovitis in individuals at risk of rheumatoid arthritis. For some individuals, ultrasound evidence of synovitis is present in ACPA-positive individuals without clinical arthritis and its presence is associated with future rheumatoid arthritis development (van de Stadt, Bos et al. 2010, Nam, Hensor et al. 2016). Seropositive individuals with CSA and positive findings on macrophage positron emission tomography (PET scan) may also develop rheumatoid arthritis (Gent, Voskuyl et al. 2012). However, for a proportion of individuals with CSA, including patients with CSA who are known to eventually develop rheumatoid arthritis, imaging may fail to reveal subclinical synovitis. All these observations are designed to help identify the window of opportunity when the treatment is the most effective and the least expensive. MBDA (Vectra) has also been shown to be an objective test, superior to CRP and DAS28 as predictor of radiologic progression in early rheumatoid arthritis, based on a large US study (Segurado and Sasso 2014). Positive ultrasound predicts progression to rheumatoid arthritis if ACPA are present and the individual has non-specific musculoskeletal symptoms even without clinical synovitis (Nam, Hensor et al. 2016). An extensive review (van Nies, Krabben et al. 2014) provided strong evidence accumulated on the association between symptom duration and radiologic progression.

The knowledge accumulated about the pre-clinical evolution of rheumatoid arthritis supports giving priority to active screening to identify at-risk subjects. By identifying individuals at risk by the methods disclosed herein, a plan for treatment may be initiated. Early treatment and adherence to treatment protocol improves the likelihood of remission (Stouten, Westhovens et al. 2019). Sustained treatment-free remission is an ultimate goal (Ajeganova and Huizinga 2017). Accordingly, two factors contribute to successful treatment of rheumatoid arthritis: early discovery and intensive use of all classes of DMARDs. Sustained remission and, in particular, drug-free sustained remission offer hope that early identification of patients with rheumatoid arthritis, early improved novel treatments and treatment to target to achieve remission may potentially transform the progressive course of rheumatoid arthritis disease and disrupt rheumatoid arthritis chronicity. Reports indicate that DMARD-free remission could be achieved frequently with early treatment (Burgers, Raza et al. 2019) and offers hope that early discovery and intensive treatment may have the potential to restore tolerance in rheumatoid arthritis. Treatment with inexpensive synthetic DMARD and steroids was shown to achieve this goal in some patients (Ajeganova, van Steenbergen et al. 2016). Patients may transition between clinical states before clinical manifestation of rheumatoid arthritis.

In addition, genetic and environmental risk factors predate the development of autoimmunity. In seropositive patients, the development of autoantibodies can be present for up to a decade before symptoms emerge (Jonsson, Arinbjarnarson et al. 1995, Jonsson, Thorsteinsson et al. 2000, Rantapaa-Dahlqvist, de Jong et al. 2003, Nielen, van Schaardenburg et al. 2004, Ferucci, Majka et al. 2005, Bhatia, Majka et al. 2007, Tracy, Buckley et al. 2017, Kelmenson, Wagner et al. 2019). Again, early discovery and elimination of as many modifiable health and environmental risk factors as possible increase the probabilities of treating rheumatoid arthritis.

Individuals at risk of rheumatoid arthritis may progress to develop symptoms, but without clinical arthritis. This phase has been termed “clinically suspect arthralgia” (CSA) when a rheumatologist has a high index of suspicion for the development of future clinical joint swelling and subsequently rheumatoid arthritis (van Steenbergen, Aletaha et al. 2017). Again, timely treatment can significantly alter disease progression and outcome (Finckh, Liang et al. 2006, Finckh, Bansback et al. 2009, Finckh, Bansback et al. 2010, van der Linden, le Cessie et al. 2010, Contreras-Yanez and Pascual-Ramos 2015). For example, methotrexate has been shown to delay the onset of rheumatoid arthritis patients (van Dongen, van Aken et al. 2007). Combination treatment of synthetic DMARDs to delay onset has not been attempted despite being significantly more effective than methotrexate alone. In general, more treatment provides better results in rheumatoid arthritis. In 70% of patients, remission is not achieved with methotrexate monotherapy alone. But with triple therapy, 30% of patients do achieve remission (Saunders, Capell et al. 2008).

Prior approaches to identify individuals at risk of rheumatoid arthritis have taken a number of different forms. First-degree relatives of patients with rheumatoid arthritis were evaluated for rheumatoid arthritis related autoantibodies and symptoms. Patients were also identified that presented with musculoskeletal symptoms and the risk for rheumatoid arthritis was quantified on the basis of symptoms and the results of laboratory and imaging tests (Nielen, van Schaardenburg et al. 2004, Nielen, van Schaardenburg et al. 2004, Nielen, van der Horst et al. 2005, Van De Stadt, Witte et al. 2013, Tracy, Buckley et al. 2017). A number of quantitative studies have been undertaken to explore the symptoms in patients at risk of rheumatoid arthritis and relate these to future rheumatoid arthritis development. Survey questions are largely based on symptoms characteristic of established rheumatoid arthritis and are therefore assumed to be present in at-risk individuals as well (Stack, Sahni et al. 2013). Common clinical manifestations in symptomatic patients prior to the development of joint swelling include symmetrical pain affecting the upper and lower extremities (van de Stadt, Witte et al. 2013, Rakieh, Nam et al. 2015, van Steenbergen, Mangnus et al. 2016), in particular, the small joints of the hands. A great proportion of those with early morning stiffness that lasted more than 60 minutes went on to develop inflammatory arthritis. A cross sectional analysis conducted on a Dutch cohort suggested that increased early morning stiffness correlates with rheumatoid arthritis development in symptomatic ‘at-risk’ patients (van Nies, van Steenbergen et al. 2015).

To date, no mass screening efforts have been undertaken due to logistics, sensitivity of the tests, and prohibitive costs of venous blood collection and/or blood processing. Clearly, the pre-test probability of rheumatoid arthritis development and thus the predictive value of specific risk factors in these scenarios are different (Suter, Fraenkel et al. 2006, Sheppard, Kumar et al. 2008). With the methods disclosed herein, however, mass screening of populations for rheumatoid arthritis or other autoimmune diseases is possible. According to certain aspects of this disclosure, the unique blood stabilization solution facilitates the ability to suspend and preserve blood samples containing biomarkers for rheumatoid arthritis and other diseases. The inventors unexpectedly and surprisingly discovered that the specific components of the blood stabilization solution provided a means to preserve individual whole blood samples for nearly a week.

According to the methods described herein, a general population may be screened for various predictive factors for rheumatoid arthritis. The screening may include filling out a questioner, answering a series of questions via a computer database, conducting a person to person interview, etc. Individuals may be identified as high risk based upon the screening data and evaluation of risk factors such as medical history, gender, age, race, joint pain history, alcohol consumption, smoking, body weight/height, stress, diet, periodontal disease, family history, etc. The screening data may be processed by a computing device and related software algorithms or instructions to determine if an individual is at risk. Predictive algorithms including demographic, clinical and laboratory variables, such as bone edema on MM for example, have previously been developed for predicting the development of rheumatoid arthritis in patients with autoantibody-positive arthralgia and undifferentiated arthritis (van der Helm-van Mil, Detert et al. 2008, Duer-Jensen, Horslev-Petersen et al. 2011). Patients with seropositive arthralgia, symptoms of recent onset, affected upper and lower extremities, and associated with more than one hour of early morning stiffness, identified those more likely to progress to rheumatoid arthritis (van de Stadt, Witte et al. 2013). Similarly, symmetrical symptoms affecting the upper and lower extremities with severe morning stiffness increased the likelihood of patients developing rheumatoid arthritis (De Rooy, Van Der Linden et al. 2011). Other studies also noted the rheumatoid arthritis symptoms without joint swelling (Jutley, Latif et al. 2017)]. Other risk values of interest include boy mass index, smoking status, duration and nature and progression of general symptoms, morning stiffness, small joint symptoms, symmetry, and upper limb involvement, etc. (Norli, Brinkmann et al. 2017). Notably, high body mass index predicts less remission and less sustained remission in early rheumatoid arthritis, indicating the need for patients at risk for developing rheumatoid arthritis, based on serology, to lose weight (Schulman, Bartlett et al. 2018). Relatively high body mass index in seropositive patients increases the risk of rheumatoid arthritis based on meta-analysis of 16 studies that included 406,584 participants (Feng, Xu et al. 2019). The studies also noted that bariatric surgery does not seem to have any benefit (Sparks, Halperin et al. 2015).

By processing the screening data and identifying an individual at high risk, collection of a blood sample would be required to confirm whether or not an individual is likely of developing rheumatoid arthritis or other autoimmune disease. By using the methods disclosed herein, a small amount of blood (e.g., 20 μL) could be collected by finger prick in virtually any setting such as an individual's home, doctors office, urgent care facility, drug store, etc. The collected blood sample and related biomarkers could subsequently be processed, analyzed, and compared to the screening data to determining if the individual is at high risk for developing rheumatoid arthritis or other autoimmune disease based on the biomarker type(s) and the biomarker level(s). An individual identified as high risk by the methods discussed herein may begin treatment with a therapeutic amount of any of the drugs disclosed herein, to include non-steroidal anti-inflammatory drugs, synthetic disease-modifying anti-rheumatic drugs (DMARDS), and biological DMARDS.

By stabilizing and preserving the whole blood samples by the methods disclosed herein, collection and processing times are preserved resulting in a decrease in expense. Thus, mass screening populations becomes possible due to the reduced cost and ability to preserve the whole blood samples for at least a week. Accordingly, kits for the mass rheumatoid arthritis screening or other autoimmune disease screen may be distributed to various facilities at significantly reduced costs compared to current methods and procedures.

### EXAMPLE 1

Peripheral blood with ethylene diamine tetra acetic acid (EDTA) as anti-coagulant was collected from five patients. The blood was diluted 1:100 in a blood stabilization and preservative solution (also known as Therazyme™ or “TZ”) containing Heparin. Table 2 below describes the components of the blood stabilizing solution. The whole blood may be collected via finger prick or other suitable method known in the art. The whole blood may be transferred to an appropriate collection vial that is prelabeled with a barcode or other suitable computer readable label. In some examples, the blood sample is at least 1 μL, 2 μL, 3 μL, 4 μL, 5 μL, 6 μL, 7 μL, 8 μL, 9 μL, 10 μL, 11 μL, 12 μL, 13 μL, 14 μL, 15 μL, 16 μL, 17 μL, 18 μL, 19 μL, 20 μL, 21 μL, 22 μL, 23 μL, 24 μL, 25 μL, 26 μL, 27 μL, 28 μL, 28 μL, 29 μL, 30 μL, 31 μL, 32 μL, 33 μL, 34 μL, 35 μL, 36 μL, 37 μL, 38 μL, 39 μL, 40 μL, 41 μL, 42 μL, 43 μL, 44 μL, 45 μL, 46 μL, 47 μL, 48 μL, 49 μL, 50 μL, 10-50 μL, 50-75 μL, 75-100, or 5-100 μL of whole blood.

TABLE 2

| Material | Manufacturer | Concentration |
|---|---|---|
| Tris buffered saline (10x) | TheraTest Labs | 1:10 |
| Bovine serum albumin | Sigma | 0.5% |
| Tyrosine | Sigma | 0.04% |
| CaCl | Sigma | 0.05% |
| Trehalose | Sigma | 1.0% |
| ProClin 950 | Sigma | 1:1000 |
| DI Water | TheraTest Labs | Up to Volume |

In some examples, the amount of blood stabilizing solution is a level necessary to preserve the biomarkers of the sample. In other examples, the solution may include tris-HC1 in buffered saline, bovine serum albumin, tyrosine, calcium chloride, trehalose, a preservative, and water. In some examples, concentration of the bovine serum albumin in the solution is at least 0.1%, 0.2%, 0.3%, 0.4%, 0.5%, 0.6%, 0.7%, 0.8%, 0.9%, or 1.0%. In other examples, the concentration of the bovine serum albumin in the solution is about 0.1% to 0.5%, 0.5% to 1.0%, 1.0% to 1.5%, or 0.1% to 1.5%. In still other examples, concentration of the tris-HC1 in buffered saline is at least 0.05M, 0.06M, 0.07M, 0.08M, 0.09M, 0.10M, 0.11M, 0.12M, 0.13M, 0.14M, 0.15M, 0.16M, 0.17M, 0.18M, 0.19M, or 0.20M. In some examples, concentration of the tris-HC1 in buffered saline is about 0.01M to 1.0M. In other examples, the concentration of the tyrosine is at least 0.1%, 0.2%, 0.3%, 0.4%, 0.5%, 0.6%, 0.7%, 0.8%, 0.9%, or 1.0%. In still other examples, the concentration of the tyrosine is about 0.1% to 0.5%, 0.5% to 1.0%, 1.0% to 1.5%, or 0.1% to 1.5%. In some examples, concentration of the calcium chloride in the solution is at least 0.01%, 0.02%, 0.03%, 0.04%, 0.05%, 0.06%, 0.07%, 0.08%, 0.09%, or 0.1%. In other examples, the concentration of the calcium chloride in the solution is about 0.01% to 0.05%, 0.05% to 0.1%, 0.1% to 1.0%, or 0.1% to 1.5%. In other examples, the concentration of the trehalose is at least 0.1%, 0.2%, 0.3%, 0.4%, 0.5%, 0.6%, 0.7%, 0.8%, 0.9%, 1.0%, 2.0%, 3.0%, 4.0%, 5.0%, 6.0%, 7.0%, 8.0%, 9.0%, or 10.0%. In still other examples, the concentration of the trehalose is about 0.1% to 1.0%, 1.0% to 5.0%, 5.0% to 10.0%, or 0.1% to 10.0%. In some examples, the concentration of the preservative is at least 0.001%, 0.002%, 0.003%, 0.004%, 0.005%, 0.006%, 0.007%, 0.008%, 0.009%, 0.01%, 0.1%, 0.2%, 0.3%, 0.4%, 0.5%, 0.6%, 0.7%, 0.8%, 0.9%, 1.0%, or 10.0%. In some examples, the preservative may be 2-methyl-4-isothiazolin-3-one solution.

[54] The blood sample was either tested as is or spiked with a known positive serum sample

RF011 at a 1:4700 final dilution for measuring antibodies to both IgM and IgA (RA) isotypes, or at 1:500 final dilution for measuring antibodies to cyclic citrullinated peptide. The spiked RF011 serum in TherazymeTM (TZ)-Heparin alone served as a positive control. See Table 2 below.

TABLE 3

| Sample Number | Therazyme ™ | RF011 | Blood |
|---|---|---|---|
| 1 | + | + | − |
| 2 | + | + | + |
| 3 | + | − | + |

The samples were incubated at room temp for 2.5-3 hours to allow the red blood cells and the white blood cells to settle at the bottom of the collection tube/vial. IgM and IgA RF ELISA and cCP2-IgG and IgA ELISA were the performed on supernatant (diluted plasma) at different time points to include 2 hours, overnight, 2 days and 7 days (samples were incubated at 4° C. in between testing). At each point, supernatant was directly tested from the tube with the red blood cell and white blood cell pellet at the bottom of the tube. Data is shown in FIGS. 2 and 3.

FIG. 1 indicates the identified rheumatoid arthritis biomarker levels for IgM and IgA RF, over various time periods, in the diluted whole blood samples suspended and preserved by the stabilizing solution as described herein. Results for the patients—S28 through S32—are shown in FIG. 1. The first sample, as shown in Table 3, is spiked positive control serum diluted in TZ+Heparin. The second sample is spiked positive control serum diluted in TZ+Heparin in presence of the patient's blood, and the third sample is the patient's blood diluted in TZ+Heparin over different time points. The inventors discovered, surprisingly, that the stabilizing solution as described herein successfully preserved the biomarkers, RF IgM and IgA, so that the biomarkers were still detectable and identifiable after 7 days.

FIG. 2 indicates the identified the rheumatoid arthritis biomarker levels for cyclic citrullinated peptide 2 (cCP2), over various time periods, in the diluted whole blood samples suspended and preserved by the stabilizing solution described herein. Again, the first sample, as shown in Table 3, is spiked positive control serum diluted in TZ+Heparin. The second sample is spiked positive control serum diluted in TZ+Heparin in presence of the patient's blood, and the third sample is the patient's blood diluted in TZ+Heparin over different time points. Once again, the inventors discovered, surprisingly, that the stabilizing solution as described herein successfully preserved the biomarker, cyclic citrullinated peptide, so that the biomarker was still detectable and identifiable after 7 days.

Accordingly, the methods disclosed herein may preserve a biomarker for testing and analysis for at least 1 day, 2, days, 3 days, 4 days, 5 days, 6 days, 7 days, 8 days, 9 days, or 10 days. In other examples, the methods disclosed herein may preserve a biomarker for testing and analysis for at least 4 hours, 6, hours, 8 hours, 10 hours, 12, hours, 24 hours, 36 hours, 48 hours, 60 hours, 72 hours, 96 hours, 120 hours, 144 hours, or 168 hours.

[60] One or more aspects or screening, analyzing, and determining an individual's risk discussed herein may be embodied in computer-usable or readable data and/or computer-executable instructions, such as in one or more program modules, executed by one or more computers or other devices as described herein. Generally, program modules include routines, programs, objects, components, data structures, and the like that perform particular tasks or implement particular abstract data types when executed by a processor in a computer or other device. The modules may be written in a source code programming language that is subsequently compiled for execution, or may be written in a scripting language such as (but not limited to) HTML, or XML. The computer executable instructions may be stored on a computer readable medium such as a hard disk, optical disk, removable storage media, solid-state memory, RAM, and the like. As will be appreciated by one of skill in the art, the functionality of the program modules may be combined or distributed as desired in various embodiments. In addition, the functionality may be embodied in whole or in part in firmware or hardware equivalents such as integrated circuits, field programmable gate arrays (FPGA), and the like. Particular data structures may be used to more effectively implement one or more aspects discussed herein, and such data structures are contemplated within the scope of computer executable instructions and computer-usable data described herein. Various aspects discussed herein may be embodied as a method, a computing device, a system, and/or a computer program product.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The present disclosure is disclosed above and in the accompanying drawings with reference to a variety of examples. The purpose served by the disclosure, however, is to provide examples of the various features and concepts related to the disclosure, not to limit the scope of the invention. One skilled in the relevant art will recognize that numerous variations and modifications may be made to the examples described above without departing from the scope of the present disclosure.

### REFERENCES

The references listed below are all hereby incorporated herein by reference in their entirety for all purposes.

Aho, K., M. Heliovaara, J. Maatela, T. Tuomi and T. Palosuo (1991). “Rheumatoid factors antedating clinical rheumatoid arthritis.” J Rheumatol 18(9): 1282-1284.

Ajeganova, S. and T. Huizinga (2017). “Sustained remission in rheumatoid arthritis: latest evidence and clinical considerations.” Ther Adv Musculoskelet Dis 9(10): 249-262.

Ajeganova, S., H. W. van Steenbergen, J. A. van Nies, L. E. Burgers, T. W. Huizinga and A. H. van der Helm-van Mil (2016). “Disease-modifying antirheumatic drug-free sustained remission in rheumatoid arthritis: an increasingly achievable outcome with subsidence of disease symptoms.” Ann Rheum Dis 75(5): 867-873.

Aletaha, D., T. Neogi, A. J. Silman, J. Funovits, D. T. Felson, C. O Bingham, 3rd, N. S.

Birnbaum, G. R. Burmester, V. P. Bykerk, M. D. Cohen, B. Combe, K. H. Costenbader, M. Dougados, P. Emery, G. Ferraccioli, J. M. Hazes, K. Hobbs, T. W. Huizinga, A. Kavanaugh, J. Kay, T. K. Kvien, T. Laing, P. Mease, H. A. Menard, L. W. Moreland, R. L. Naden, T. Pincus, J. S. Smolen, E. Stanislawska-Biernat, D. Symmons, P. P. Tak, K. S. Upchurch, J. Vencovsky, F. Wolfe and G. Hawker (2010). “2010 rheumatoid arthritis classification criteria: an American College of Rheumatology/European League Against Rheumatism collaborative initiative.” Ann Rheum Dis 69(9): 1580-1588. Baker, K. F., A. J. Skelton, D. W. Lendrem, A. Scadeng, B. Thompson, A. G. Pratt and J. D. Isaacs (2019). “Predicting drug-free remission in rheumatoid arthritis: A prospective interventional cohort study.” J Autoimmun: 102298.

Barnabe, C., J. Xiong, J. E. Pope, G. Boire, C. Hitchon, B. Haraoui, J. Carter Thorne, D.

Tin, E. C. Keystone and V. P. Bykerk (2014). “Factors associated with time to diagnosis in early rheumatoid arthritis.” 34(1): 85-92.

Bejarano, V., P. G. Conaghan, M. A. Quinn, B. Saleem and P. Emery (2010). “Benefits 8 years after a remission induction regime with an infliximab and methotrexate combination in early rheumatoid arthritis.” Rheumatology (Oxford) 49(10): 1971-1974.

Bhatia, S. S., D. S. Majka, J. M. Kittelson, L. A. Parrish, E. D. Ferucci, K. D. Deane, W.

P. Arend, M. Rewers, V. Michael Holers and J. M. Norris (2007). “Rheumatoid factor seropositivity is inversely associated with oral contraceptive use in women without rheumatoid arthritis.” Ann Rheum Dis 66(2): 267-269.

Boeters, D. M., W. P. Nieuwenhuis, M. K. Verheul, E. C. Newsum, M. Reijnierse, R. E.

Toes, L. A. Trouw and A. H. van der Helm-van Mil (2016). “MRI-detected osteitis is not associated with the presence or level of ACPA alone, but with the combined presence of ACPA and RF.” Arthritis Res Ther 18: 179.

Bos, W. H., G. J. Wolbink, M. Boers, G. J. Tijhuis, N. de Vries, I. E. van der Horst-Bruinsma, P. P. Tak, R. J. van de Stadt, C. J. van der Laken, B. A. Dijkmans and D. van Schaardenburg (2010). “Arthritis development in patients with arthralgia is strongly associated with anti-citrullinated protein antibody status: a prospective cohort study.” Ann Rheum Dis 69(3): 490-494.

Brasington, R. D. J., Miner,J.J. (2019). Clinical features of rheumatoid arthritis.

Philadelphia, Pa., Elsevier.

Brink, M., M. Hansson, L. Mathsson-Alm, P. Wijayatunga, M. K. Verheul, L. A. Trouw,

R. Holmdahl, J. Ronnelid, L. Klareskog and S. Rantapaa-Dahlqvist (2016). “Rheumatoid factor isotypes in relation to antibodies against citrullinated peptides and carbamylated proteins before the onset of rheumatoid arthritis.” Arthritis Res Ther 18: 43.

Burgers, L. E., K. Raza and A. H. Van Der Helm-Van Mil (2019). “Window of opportunity in rheumatoid arthritis—definitions and supporting evidence: from old to new perspectives.” RMD Open 5(1): e000870.

Cader, M. Z., A. Filer, J. Hazlehurst, P. de Pablo, C. D. Buckley and K. Raza (2011). “Performance of the 2010 ACR/EULAR criteria for rheumatoid arthritis: comparison with 1987 ACR criteria in a very early synovitis cohort.” Ann Rheum Dis 70(6): 949-955.

Cheng, Z.., J. Meade, K. Mankia, P. Emery and D. A. Devine (2017). “Periodontal disease and periodontal bacteria as triggers for rheumatoid arthritis.” Best Pract Res Clin Rheumatol 31(1): 19-30.

Chibnik, L. B., L. A. Mandl, K. H. Costenbader, P. H. Schur and E. W. Karlson (2009).

“Comparison of threshold cutpoints and continuous measures of anti-cyclic citrullinated peptide antibodies in predicting future rheumatoid arthritis.” J Rheumatol 36(4): 706-711.

Contreras-Yanez, I. and V. Pascual-Ramos (2015). “Window of opportunity to achieve major outcomes in early rheumatoid arthritis patients: how persistence with therapy matters.” Arthritis Res Ther 17: 177.

Cope, A. P. (2017). “Emerging therapies for pre-RA.” Best Pract Res Clin Rheumatol 31(1): 99-111.

De Rooy, D. P. C., M. P. M. Van Der Linden, R. Knevel, T. W. J. Huizinga and A. H. M. Van Der Helm-Van Mil (2011). “Predicting arthritis outcomes--what can be learned from the Leiden Early Arthritis Clinic?” Rheumatology 50(1): 93-100.

De Winter, L. M., W. L. Hansen, H. W. van Steenbergen, P. Geusens, J. Lenaerts, K. Somers, P. Stinissen, A. H. van der Helm-van Mil and V. Somers (2016). “Autoantibodies to two novel peptides in seronegative and early rheumatoid arthritis.” Rheumatology (Oxford) 55(8): 1431-1436.

Deane, K. D., M. K. Demoruelle, L. B. Kelmenson, K. A. Kuhn, J. M. Norris and V. M. Holers (2017). “Genetic and environmental risk factors for rheumatoid arthritis.” Best Pract Res Clin Rheumatol 31(1): 3-18.

del Puente, A., W. C. Knowler, D. J. Pettitt and P. H. Bennett (1988). “The incidence of rheumatoid arthritis is predicted by rheumatoid factor titer in a longitudinal population study.” Arthritis Rheum 31(10): 1239-1244.

Del Puente, A., W. C. Knowler, D. J. Pettitt and P. H. Bennett (1989). “High incidence and prevalence of rheumatoid arthritis in Pima Indians.” Am J Epidemiol 129(6): 1170-1178.

Duer-Jensen, A., K. Horslev-Petersen, M. L. Hetland, L. Bak, B. J. Ejbjerg, M. S. Hansen, J. S. Johansen, H. M. Lindegaard, H. Vinterberg, J. M. Moller and M. Ostergaard (2011). “Bone edema on magnetic resonance imaging is an independent predictor of rheumatoid arthritis development in patients with early undifferentiated arthritis.” Arthritis Rheum 63(8): 2192-2202.

Emery, P., K. Mankia and J. L. Nam (2017). “Individuals at risk of rheumatoid arthritis—The evolving story.” Best Pract Res Clin Rheumatol 31(1): 1-2.

Falkenburg, W. J. J. and D. van Schaardenburg (2017). “Evolution of autoantibody responses in individuals at risk of rheumatoid arthritis.” Best Pract Res Clin Rheumatol 31(1): 42-52.

Feng, X., X. Xu, Y. Shi, X. Liu, H. Liu, H. Hou, L. Ji, Y. Li, W. Wang, Y. Wang and D. Li (2019). “Body Mass Index and the Risk of Rheumatoid Arthritis: An Updated Dose-Response Meta-Analysis.” Biomed Res Int 2019: 3579081.

Ferucci, E. D., D. S. Majka, L. A. Parrish, M. B. Moroldo, M. Ryan, M. Passo, S. D. Thompson, K. D. Deane, M. Rewers, W. P. Arend, D. N. Glass, J. M. Norris and V. M. Holers (2005). “Antibodies against cyclic citrullinated peptide are associated with HLA-DR4 in simplex and multiplex polyarticular-onset juvenile rheumatoid arthritis.” Arthritis Rheum 52(1): 239-246.

Finckh, A., N. Bansback and M. H. Liang (2010). “Cost-effectiveness of biologics in early rheumatoid arthritis.” Ann Intern Med 152(5): 333-334; author reply 334.

Finckh, A., N. Bansback, C. A. Marra, A. H. Anis, K. Michaud, S. Lubin, M. White, S. Sizto and M. H. Liang (2009). “Treatment of very early rheumatoid arthritis with symptomatic therapy, disease-modifying antirheumatic drugs, or biologic agents: a cost-effectiveness analysis.” Ann Intern Med 151(9): 612-621.

Finckh, A., M. H. Liang, C. M. van Herckenrode and P. de Pablo (2006). “Long-term impact of early treatment on radiographic progression in rheumatoid arthritis: A meta-analysis.” Arthritis Rheum 55(6): 864-872.

Gabriel, S. E., C. S. Crowson and W. M. O'Fallon (1999). “Mortality in rheumatoid arthritis: have we made an impact in 4 decades?” J Rheumatol 26(12): 2529-2533.

Gan, R. W., L. A. Trouw, J. Shi, R. E. Toes, T. W. Huizinga, M. K. Demoruelle, J. R. Kolfenbach, G. 0. Zerbe, K. D. Deane, J. D. Edison, W. R. Gilliland, J. M. Norris and V. M. Holers (2015). “Anti-carbamylated protein antibodies are present prior to rheumatoid arthritis and are associated with its future diagnosis.” J Rheumatol 42(4): 572-579.

Gan, R. W., L. A. Trouw, J. Shi, R. E. M. Toes, T. W. J. Huizinga, M. K. Demoruelle, J. R. Kolfenbach, G. 0. Zerbe, K. D. Deane, J. D. Edison, W. R. Gilliland, J. M. Norris and V. M. Holers (2015). “Anti-carbamylated Protein Antibodies Are Present Prior to Rheumatoid Arthritis and Are Associated with Its Future Diagnosis.” The Journal of Rheumatology 42(4): 572-579.

Gent, Y. Y., A. E. Voskuyl, R. W. Kloet, D. van Schaardenburg, 0. S. Hoekstra, B. A. Dijkmans, A. A. Lammertsma and C. J. van der Laken (2012). “Macrophage positron emission tomography imaging as a biomarker for preclinical rheumatoid arthritis: findings of a prospective pilot study.” Arthritis Rheum 64(1): 62-66.

Hedström, A. K., J. Ronnelid, L. Klareskog and L. Alfredsson (2019). “Complex Relationships of Smoking, HLA-DRB1 Genes, and Serologic Profiles in Patients With Early Rheumatoid Arthritis: Update From a Swedish Population-Based Case-Control Study.” Arthritis & Rheumatology 71(9): 1504-1511.

Horta-Baas, G., M. D. S. Romero-Figueroa, A. J. Montiel-Jarquin, M. L. Pizano-Zárate, J. Garc ía-Mena and N. Ramirez-Duran (2017). “Intestinal Dysbiosis and Rheumatoid Arthritis: A Link between Gut Microbiota and the Pathogenesis of Rheumatoid Arthritis.” Journal of Immunology Research 2017: 1-13.

Houssien, D. A., T. Jonsson, E. Davies and D. L. Scott (1998). “Rheumatoid factor isotypes, disease activity and the outcome of rheumatoid arthritis: comparative effects of different antigens.” Scand J Rheumatol 27(1): 46-53.

Hresko, A., T.-C. Lin and D. H. Solomon (2018). “Medical Care Costs Associated With Rheumatoid Arthritis in the US: A Systematic Literature Review and Meta-Analysis.” Arthritis Care & Research 70(10): 1431-1438.

Hyrich, K. L. (2008). “Patients with suspected rheumatoid arthritis should be referred early to rheumatology.” BMJ 336(7637): 215-216.

Ioan-Facsinay, A., A. Willemze, D. B. Robinson, C. A. Peschken, J. Markland, D. van der Woude, B. Elias, H. A. Menard, M. Newkirk, M. J. Fritzler, R. E. Toes, T. W. Huizinga and H. S. El-Gabalawy (2008). “Marked differences in fine specificity and isotype usage of the anti-citrullinated protein antibody in health and disease.” Arthritis Rheum 58(10): 3000-3008.

James, J. A., H. Chen, K. A. Young, E. A. Bemis, J. Seifert, R. L. Bourn, K. D. Deane, M. K. Demoruelle, M. Feser, J. R. O'Dell, M. H. Weisman, R. M. Keating, P. M. Gaffney, J. A. Kelly, C. D. Langefeld, J. B. Harley, W. Robinson, D. A. Hafler, K. C. O'Connor, J. Buckner, J. M. Guthridge, J. M. Norris and V. M. Holers (2019). “Latent autoimmunity across disease-specific boundaries in at-risk first-degree relatives of SLE and RA patients.” EBioMedicine 42: 76-85.

Jiang, X., T. Frisell, J. Askling, E. W. Karlson, L. Klareskog, L. Alfredsson and H. Kallberg (2015). “To What Extent Is the Familial Risk of Rheumatoid Arthritis Explained by Established Rheumatoid Arthritis Risk Factors?” Arthritis & Rheumatology 67(2): 352-362.

Jonsson, T., S. Arinbjarnarson, J. Thorsteinsson, K. Steinsson, A. J. Geirsson, H. Jonsson and H. Valdimarsson (1995). “Raised IgA rheumatoid factor (RF) but not IgM RF or IgG RF is associated with extra-articular manifestations in rheumatoid arthritis.” Scand J Rheumatol 24(6): 372-375.

Jonsson, T., H. Thorsteinsson, S. Arinbjarnarson, J. Thorsteinsson and H. Valdimarsson (1995). “Clinical implications of IgA rheumatoid factor subclasses.” Ann Rheum Dis 54(7): 578-581.

Jonsson, T., J. Thorsteinsson, A. Kolbeinsson, E. Jonasdottir, N. Sigfusson and H. Valdimarsson (1992). “Population study of the importance of rheumatoid factor isotypes in adults.” Annals of the Rheumatic Diseases 51(7): 863-868.

Jonsson, T., J. Thorsteinsson and H. Valdimarsson (1992). “Rheumatoid factor isotypes and cancer prognosis.” Cancer 69(8): 2160-2165.

Jonsson, T., J. Thorsteinsson and H. Valdimarsson (2000). “Elevation of only one rheumatoid factor isotype is not associated with increased prevalence of rheumatoid arthritis-a population based study.” Scand J Rheumatol 29(3): 190-191.

Jonsson, T. and H. Valdimarsson (1998). “What about IgA rheumatoid factor in rheumatoid arthritis?” Ann Rheum Dis 57(1): 63-64.

Joshua, V., K. Chatzidionisyou and A. I. Catrina (2017). “Role of the lung in individuals at risk of rheumatoid arthritis.” Best Pract Res Clin Rheumatol 31(1): 31-41.

Juge, P.-A., J. S. Lee, E. Ebstein, H. Furukawa, E. Dobrinskikh, S. Gazal, C.

Kannengiesser, S. Ottaviani, S. Oka, S. Tohma, N. Tsuchiya, J. Rojas-Serrano, M. I. González-Perez, M. Mejía, I. Buendia-Roldán, R. Falfán-Valencia, E. Ambrocio-Ortiz, E. Manali, S. A. Papiris, T. Karageorgas, D. Boumpas, K. Antoniou, C. H. M. Van Moorsel, J. Van Der Vis, Y. A. De Man, J. C. Grutters, Y. Wang, R. Borie, L. Wemeau-Stervinou, B. Wallaert, R.-M. Flipo, H. Nunes, D. Valeyre, N. Saidenberg-Kermanac'H, M.-C.

Boissier, S. Marchand-Adam, A. Frazier, P. Richette, Y. Allanore, J. Sibilia, C. Dromer, C. Richez, T. Schaeverbeke, H. Lioté, G. Thabut, N. Nathan, S. Amselem, M. Soubrier, V. Cottin, A. Clément, K. Deane, A. D. Walts, T. Fingerlin, A. Fischer, J. H. Ryu, E. L. Matteson, T. B. Niewold, D. Assayag, A. Gross, P. Wolters, M. I. Schwarz, M. Holers, J. J. Solomon, T. Doyle, I. 0. Rosas, C. Blauwendraat, M. A. Nalls, M.-P. Debray, C. Boileau, B. Crestani, D. A. Schwartz and P. Dieu& (2018). “MUCSB Promoter Variant and Rheumatoid Arthritis with Interstitial Lung Disease.” New England Journal of Medicine 379(23): 2209-2219.

Jutley, G., K. Raza and C. D. Buckley (2015). “New pathogenic insights into rheumatoid arthritis.” Curr Opin Rheumatol 27(3): 249-255.

Jutley, G. S., Z. P. Latif and K. Raza (2017). “Symptoms in individuals at risk of rheumatoid arthritis.” Best Pract Res Clin Rheumatol 31(1): 59-70.

Kelmenson, L. B., B. D. Wagner, B. K. McNair, A. Frazer-Abel, K. M. Demoruelle, D. T. Bergstedt, M. L. Feser, L. K. Moss, M. C. Parish, E. A. Mewshaw, V. T. R. Mikuls, J. D. Edison, V. M. Holers and K. D. Deane (2019). “Timing of elevations of autoantibody isotypes in rheumatoid arthritis prior to disease diagnosis.” Arthritis Rheumatol.

Kim, K., X. Jiang, J. Cui, B. Lu, K. H. Costenbader, J. A. Sparks, S.-Y. Bang, H.-S. Lee, Y. Okada, S. Raychaudhuri, L. Alfredsson, S.-C. Bae, L. Klareskog and E. W. Karlson (2015). “Interactions Between Amino Acid-Defined Major Histocompatibility Complex Class II Variants and Smoking in Seropositive Rheumatoid Arthritis.” 67(10): 2611-2623.

Kolfenbach, J. R., K. D. Deane, L. A. Derber, C. I. O'Donnell, W. R. Gilliland, J. D. Edison, A. Rosen, E. Darrah, J. M. Norris and V. M. Holers (2010). “Autoimmunity to peptidyl arginine deiminase type 4 precedes clinical onset of rheumatoid arthritis.” Arthritis & Rheumatism 62(9): 2633-2639.

Krabben, A., W. Stomp, D. M. van der Heij de, J. A. van Nies, J. L. Bloem, T. W. Huizinga, M. Reijnierse and A. H. van der Helm-van Mil (2013). “MM of hand and foot joints of patients with anticitrullinated peptide antibody positive arthralgia without clinical arthritis.” Ann Rheum Dis 72(9): 1540-1544.

Kvalvik, A. G., M. A. Jones and D. P. Symmons (2000). “Mortality in a cohort of Norwegian patients with rheumatoid arthritis followed from 1977 to 1992.” Scand J Rheumatol 29(1): 29-37.

Lard, L. R., H. Visser, I. Speyer, I. E. vander Horst-Bruinsma, A. H. Zwinderman, F. C. Breedveld and J. M. Hazes (2001). “Early versus delayed treatment in patients with recent-onset rheumatoid arthritis: comparison of two cohorts who received different treatment strategies.” Am J Med 111(6): 446-451.

Leslie, D., P. Lipsky and A. L. Notkins (2001). “Autoantibodies as predictors of disease.” Journal of Clinical Investigation 108(10): 1417-1422.

Liu, X., S. K. Tedeschi, M. Barbhaiya, C. L. Leatherwood, C. B. Speyer, B. Lu, K. H. Costenbader, E. W. Karlson and J. A. Sparks (2019). “Impact and Timing of Smoking Cessation on Reducing Risk of Rheumatoid Arthritis Among Women in the Nurses' Health Studies.” Arthritis Care Res (Hoboken) 71(7): 914-924.

Majka, D. S., K. D. Deane, L. A. Parrish, A. A. Lazar, A. E. Baron, C. W. Walker, M. V. Rubertone, W. R. Gilliland, J. M. Norris and V. M. Holers (2008). “Duration of preclinical rheumatoid arthritis-related autoantibody positivity increases in subjects with older age at time of disease diagnosis.” Ann Rheum Dis 67(6): 801-807.

Majka, D. S. and V. M. Holers (2003). “Can we accurately predict the development of rheumatoid arthritis in the preclinical phase?” Arthritis & Rheumatism 48(10): 2701-2705.

Mankia, K. and P. Emery (2016). “Preclinical Rheumatoid Arthritis: Progress Toward Prevention.” Arthritis Rheumatol 68(4): 779-788.

Myasoedova, E., C. S. Crowson, H. M. Kremers, T. M. Therneau and S. E. Gabriel (2010). “Is the incidence of rheumatoid arthritis rising?: results from Olmsted County, Minn., 1955-2007.” Arthritis Rheum 62(6): 1576-1582.

Myasoedova, E., J. M. Davis, 3rd, C. S. Crowson and S. E. Gabriel (2010). “Epidemiology of rheumatoid arthritis: rheumatoid arthritis and mortality.” Curr Rheumatol Rep 12(5): 379-385.

Nagy, G. and R. F. Van Vollenhoven (2015). “Sustained biologic-free and drug-free remission in rheumatoid arthritis, where are we now?” Arthritis Research & Therapy 17(1).

Nam, J. L., E. M. Hensor, L. Hunt, P. G. Conaghan, R. J. Wakefield and P. Emery (2016).

“Ultrasound findings predict progression to inflammatory arthritis in anti-CCP antibody-positive patients without clinical synovitis.” Ann Rheum Dis 75(12): 2060-2067.

Newkirk, M. M., R. Goldbach-Mansky, B. W. Senior, J. Klippel, H. R. Schumacher and H. S. El-Gabalawy (2005). “Elevated levels of IgM and IgA antibodies to Proteus mirabilis and IgM antibodies to Escherichia coli are associated with early rheumatoid factor (RF)-positive rheumatoid arthritis.” Rheumatology 44(11): 1433-1441.

Nielen, M. M., A. R. van der Horst, D. van Schaardenburg, I. E. van der Horst-Bruinsma, R. J. van de Stadt, L. Aarden, B. A. Dijkmans and D. Hamann (2005). “Antibodies to citrullinated human fibrinogen (ACF) have diagnostic and prognostic value in early arthritis.” Ann Rheum Dis 64(8): 1199-1204.

Nielen, M. M., D. van Schaardenburg, H. W. Reesink, J. W. Twisk, R. J. van de Stadt, I. E. van der Horst-Bruinsma, T. de Gast, M. R. Habibuw, J. P. Vandenbroucke and B. A. Dijkmans (2004). “Increased levels of C-reactive protein in serum from blood donors before the onset of rheumatoid arthritis.” Arthritis Rheum 50(8): 2423-2427.

Nielen, M. M., D. van Schaardenburg, H. W. Reesink, R. J. van de Stadt, I. E. van der Horst-Bruinsma, M. H. de Koning, M. R. Habibuw, J. P. Vandenbroucke and B. A. Dijkmans (2004). “Specific autoantibodies precede the symptoms of rheumatoid arthritis: a study of serial measurements in blood donors.” Arthritis Rheum 50(2): 380-386.

Nielsen, S. F., S. E. Bojesen, P. Schnohr and B. G. Nordestgaard (2012). “Elevated rheumatoid factor and long term risk of rheumatoid arthritis: a prospective cohort study.” BMJ 345(sep06 2): e5244-e5244.

Nishimura, K., D. Sugiyama, Y. Kogata, G. Tsuji, T. Nakazawa, S. Kawano, K. Saigo, A. Morinobu, M. Koshiba, K. M. Kuntz, I. Kamae and S. Kumagai (2007). “Meta-analysis: diagnostic accuracy of anti-cyclic citrullinated peptide antibody and rheumatoid factor for rheumatoid arthritis.” Ann Intern Med 146(11): 797-808.

Norli, E. S., G. H. Brinkmann, T. K. Kvien, 0. Bjøorneboe, A. J. Haugen, H. Nygaard, C. Thunem, E. Lie and M. D. Mjaavatten (2017). “Diagnostic spectrum and 2-year outcome in a cohort of patients with very early arthritis.” RMD Open 3(2): e000573.

O'Dell, J. R., J. R. Curtis, T. R. Mikuls, S. S. Cofield, S. L. Bridges, Jr., V. K. Ranganath, L. W. Moreland and T. T. Investigators (2013). “Validation of the methotrexate-first strategy in patients with early, poor-prognosis rheumatoid arthritis: results from a two-year randomized, double-blind trial.” Arthritis Rheum 65(8): 1985-1994.

Okada, Y., S. Eyre, A. Suzuki, Y. Kochi and K. Yamamoto (2019). “Genetics of rheumatoid arthritis: 2018 status.” Annals of the Rheumatic Diseases 78(4): 446-453.

Quinn, M. A., P. G. Conaghan and P. Emery (2001). “The therapeutic approach of early intervention for rheumatoid arthritis: what is the evidence?” Rheumatology (Oxford) 40(11): 1211-1220.

Rakieh, C., J. L. Nam, L. Hunt, E. M. Hensor, S. Das, L. A. Bissell, E. Villeneuve, D. McGonagle, R. Hodgson, A. Grainger, R. J. Wakefield, P. G. Conaghan and P. Emery (2015). “Predicting the development of clinical arthritis in anti-CCP positive individuals with non-specific musculoskeletal symptoms: a prospective observational cohort study.” Ann Rheum Dis 74(9): 1659-1666.

Rantapaa-Dahlqvist, S., B. A. de Jong, E. Berglin, G. Hallmans, G. Wadell, H. Stenlund, U. Sundin and W. J. van Venrooij (2003). “Antibodies against cyclic citrullinated peptide and IgA rheumatoid factor predict the development of rheumatoid arthritis.” Arthritis Rheum 48(10): 2741-2749.

Raza, K., M. Breese, P. Nightingale, K. Kumar, T. Potter, D. M. Carruthers, D. Situnayake, C. Gordon, C. D. Buckley, M. Salmon and G. D. Kitas (2005). “Predictive value of antibodies to cyclic citrullinated peptide in patients with very early inflammatory arthritis.” J Rheumatol 32(2): 231-238.

Rispens, T., P. Ooievaar-De Heer, E. Vermeulen, J. Schuurman, M. Van Der Neut

Kolfschoten and R. C. Aalberse (2009). “Human IgG4 Binds to IgG4 and Conformationally Altered IgG1 via Fc-Fc Interactions.” The Journal of Immunology 182(7): 4275-4281.

Saunders, S. A., H. A. Capell, A. Stirling, R. Vallance, W. Kincaid, A. D. McMahon and D. R. Porter (2008). “Triple therapy in early active rheumatoid arthritis: A randomized, single-blind, controlled trial comparing step-up and parallel treatment strategies.” 58(5): 1310-1317.

Schellekens, G. A., B. A. de Jong, F. H. van den Hoogen, L. B. van de Putte and W. J. van Venrooij (1998). “Citrulline is an essential constituent of antigenic determinants recognized by rheumatoid arthritis-specific autoantibodies.” J Clin Invest 101(1): 273-281.

Schulman, E., S. J. Bartlett, 0. Schieir, K. M. Andersen, G. Boire, J. E. Pope, C. Hitchon, S. Jamal, J. C. Thorne, D. Tin, E. C. Keystone, B. Haraoui, S. M. Goodman and V. P. Bykerk (2018). “Overweight, Obesity, and the Likelihood of Achieving Sustained Remission in Early Rheumatoid Arthritis: Results From a Multicenter Prospective Cohort Study.” Arthritis Care & Research 70(8): 1185-1191.

Segurado, 0. G. and E. H. Sasso (2014). “Vectra DA for the objective measurement of disease activity in patients with rheumatoid arthritis.” Clin Exp Rheumatol 32(5 Suppl 85): S-29-34.

Sheppard, J., K. Kumar, C. D. Buckley, K. L. Shaw and K. Raza (2008). “‘I just thought it was normal aches and pains’: a qualitative study of decision-making processes in patients with early rheumatoid arthritis.” Rheumatology 47(10): 1577-1582.

Shi, J., L. A. van de Stadt, E. W. Levarht, T. W. Huizinga, D. Hamann, D. van Schaardenburg, R. E. Toes and L. A. Trouw (2014). “Anti-carbamylated protein (anti-CarP) antibodies precede the onset of rheumatoid arthritis.” Ann Rheum Dis 73(4): 780-783.

Sokolove, J., R. Bromberg, K. D. Deane, L. J. Lahey, L. A. Derber, P. E. Chandra, J. D. Edison, W. R. Gilliland, R. J. Tibshirani, J. M. Norris, V. M. Holers and W. H. Robinson (2012). “Autoantibody Epitope Spreading in the Pre-Clinical Phase Predicts Progression to Rheumatoid Arthritis.” PLoS ONE 7(5): e35296.

Sokolove, J., D. S. Johnson, L. J. Lahey, C. A. Wagner, D. Cheng, G. M. Thiele, K. Michaud, H. Sayles, A. M. Reimold, L. Caplan, G. W. Cannon, G. Kerr, T. R. Mikuls and W. H. Robinson (2014). “Rheumatoid factor as a potentiator of anti-citrullinated protein antibody-mediated inflammation in rheumatoid arthritis.” Arthritis Rheumatol 66(4): 813-821.

Sparks, J. A., S.-C. Chang, K. D. Deane, R. W. Gan, M. K. Demoruelle, M. L. Feser, L. Moss, J. H. Buckner, R. M. Keating, K. H. Costenbader, P. K. Gregersen, M. H. Weisman, T. R. Mikuls, J. R. O'Dell, V. M. Holers, J. M. Norris and E. W. Karlson (2016). “Associations of smoking and age with inflammatory joint signs among first-degree relatives without rheumatoid arthritis: Results from the Studies of the Etiology of RA.”

Sparks, J. A., S.-C. Chang, K. P. Liao, B. Lu, A. R. Fine, D. H. Solomon, K. H. Costenbader and E. W. Karlson (2015). “Rheumatoid arthritis and mortality among women during 36 years of prospective follow-up: Results from the Nurses' Health Study.”

Sparks, J. A., S.-C. Chang, K. P. Liao, B. Lu, A. R. Fine, D. H. Solomon, K. H. Costenbader and E. W. Karlson (2016). “Rheumatoid Arthritis and Mortality Among Women During 36 Years of Prospective Follow-Up: Results From the Nurses' Health Study.” Arthritis Care & Research 68(6): 753-762.

Sparks, J. A., C.-Y. Chen, L. T. Hiraki, S. Malspeis, K. H. Costenbader and E. W. Karlson (2014). “Contributions of Familial Rheumatoid Arthritis or Lupus and Environmental Factors to Risk of Rheumatoid Arthritis in Women: A Prospective Cohort Study.” Arthritis Care & Research 66(10): 1438-1446.

Sparks, J. A., F. Halperin, J. C. Karlson, E. W. Karlson and B. L. Bermas (2015). “Impact of Bariatric Surgery on Patients With Rheumatoid Arthritis.” 67(12): 1619-1626.

Sparks, J. A., M. D. Iversen, R. Miller Kroouze, T. G. Mahmoud, N. A. Triedman, S. S. Kalia, M. L. Atkinson, B. Lu, K. D. Deane, K. H. Costenbader, R. C. Green and E. W. Karlson (2014). “Personalized Risk Estimator for Rheumatoid Arthritis (PRE-RA) Family Study: Rationale and design for a randomized controlled trial evaluating rheumatoid arthritis risk education to first-degree relatives.” Contemporary Clinical Trials 39(1): 145-157.

Sparks, J. A., M. D. Iversen, Z. Yu, N. A. Triedman, M. G. Prado, R. Miller Kroouze, S. S. Kalia, M. L. Atkinson, E. A. Mody, S. M. Helfgott, D. J. Todd, P. F. Dellaripa, B. L. Bermas, K. H. Costenbader, K. D. Deane, B. Lu, R. C. Green and E. W. Karlson (2018). “Disclosure of Personalized Rheumatoid Arthritis Risk Using Genetics, Biomarkers, and Lifestyle Factors to Motivate Health Behavior Improvements: A Randomized Controlled Trial.” Arthritis Care & Research 70(6): 823-833.

Sparks, J. A. and E. W. Karlson (2016). “The Roles of Cigarette Smoking and the Lung in the Transitions Between Phases of Preclinical Rheumatoid Arthritis.” Current Rheumatology Reports 18(3).

Stack, R. J., M. Sahni, C. D. Mallen and K. Raza (2013). “Symptom complexes at the earliest phases of rheumatoid arthritis: a synthesis of the qualitative literature.” Arthritis Care Res (Hoboken) 65(12): 1916-1926.

Stack, R. J., K. Shaw, C. Mallen, S. Herron-Marx, R. Home and K. Raza (2012). “Delays in help seeking at the onset of the symptoms of rheumatoid arthritis: a systematic synthesis of qualitative literature.” Annals of the Rheumatic Diseases 71(4): 493-497.

Stack, R. J., L. H. van Tuyl, M. Sloots, L. A. van de Stadt, W. Hoogland, B. Maat, C. D. Mallen, R. Tiwana, K. Raza and D. van Schaardenburg (2014). “Symptom complexes in patients with seropositive arthralgia and in patients newly diagnosed with rheumatoid arthritis: a qualitative exploration of symptom development.” Rheumatology (Oxford) 53(9):1646-1653.

Stouten, V., R. Westhovens, S. Pazmino, D. De Cock, K. Van der Elst, J. Joly, P. Verschueren and R. A. s. g. Care (2019). “Effectiveness of different combinations of DMARDs and glucocorticoid bridging in early rheumatoid arthritis: two-year results of CareRA.” Rheumatology (Oxford).

Suter, L. G., L. Fraenkel and E. S. Holmboe (2006). “What factors account for referral delays for patients with suspected rheumatoid arthritis?” Arthritis & Rheumatism 55(2): 300-305.

Swedler, W., J. Wallman, C. J. Froelich and M. Teodorescu (1997). “Routine measurement of IgM, IgG, and IgA rheumatoid factors: high sensitivity, specificity, and predictive value for rheumatoid arthritis.” J Rheumatol 24(6): 1037-1044.

Symmons, D. P., M. A. Jones, D. L. Scott and P. Prior (1998). “Longterm mortality outcome in patients with rheumatoid arthritis: early presenters continue to do well.” J Rheumatol 25(6): 1072-1077.

Thiele, G. M., M. J. Duryee, D. R. Anderson, L. W. Klassen, S. M. Mohring, K. A. Young,

D. Benissan-Messan, H. Sayles, A. Dusad, C. D. Hunter, J. Sokolove, W. H. Robinson, J. R. O'Dell, A. P. Nicholas, D. J. Tuma and T. R. Mikuls (2015). “Malondialdehyde-Acetaldehyde Adducts and Anti-Malondialdehyde-Acetaldehyde Antibodies in Rheumatoid Arthritis.” Arthritis & Rheumatology 67(3): 645-655.

Tomasson, G., T. Aspelund, T. Jonsson, H. Valdimarsson, D. T. Felson and V. Gudnason (2010). “Effect of rheumatoid factor on mortality and coronary heart disease.” Ann Rheum Dis 69(9): 1649-1654.

Tracy, A., C. D. Buckley and K. Raza (2017). “Pre-symptomatic autoimmunity in rheumatoid arthritis: when does the disease start?” Seminars in Immunopathology 39(4): 423-435.

van de Stadt, L. A., W. H. Bos, M. Meursinge Reynders, H. Wieringa, F. Turkstra, C. J.

van der Laken and D. van Schaardenburg (2010). “The value of ultrasonography in predicting arthritis in auto-antibody positive arthralgia patients: a prospective cohort study.” Arthritis Res Ther 12(3): R98.

Van De Stadt, L. A., B. I. Witte, W. H. Bos and D. Van Schaardenburg (2013). “A prediction rule for the development of arthritis in seropositive arthralgia patients.” Annals of the Rheumatic Diseases 72(12): 1920-1926.

van de Stadt, L. A., B. I. Witte, W. H. Bos and D. van Schaardenburg (2013). “A prediction rule for the development of arthritis in seropositive arthralgia patients.” Ann Rheum Dis 72(12): 1920-1926.

van der Heide, A., J. W. Jacobs, J. W. Bijlsma, A. H. Heurkens, C. van Booma-Frankfort,

M. J. van der Veen, H. C. Haanen, D. M. Hofman, G. A. van Albada-Kuipers, E. J. ter Borg, H. L. Brus, H. J. Dinant, A. A. Kruize and Y. Schenk (1996). “The effectiveness of early treatment with “second-line” antirheumatic drugs. A randomized, controlled trial.” Ann Intern Med 124(8): 699-707.

van der Helm-van Mil, A. H., J. Detert, S. le Cessie, A. Filer, H. Bastian, G. R. Burmester, T. W. Huizinga and K. Raza (2008). “Validation of a prediction rule for disease outcome in patients with recent-onset undifferentiated arthritis: moving toward individualized treatment decision-making.” Arthritis Rheum 58(8): 2241-2247.

van der Linden, M. P., S. le Cessie, K. Raza, D. van der Woude, R. Knevel, T. W. Huizinga and A. H. van der Helm-van Mil (2010). “Long-term impact of delay in assessment of patients with early arthritis.” Arthritis Rheum 62(12): 3537-3546.

van Dongen, H., J. van Aken, L. R. Lard, K. Visser, H. K. Ronday, H. M. Hulsmans, I. Speyer, M. L. Westedt, A. J. Peeters, C. F. Allaart, R. E. Toes, F. C. Breedveld and T. W. Huizinga (2007). “Efficacy of methotrexate treatment in patients with probable rheumatoid arthritis: a double-blind, randomized, placebo-controlled trial.” Arthritis Rheum 56(5): 1424-1432.

van Nies, J. A., C. Alves, A. L. Radix-Bloemen, C. Gaujoux-Viala, T. W. Huizinga, J. M. Hazes, E. Brouwer, B. Fautrel and A. H. van der Helm-van Mil (2015). “Reappraisal of the diagnostic and prognostic value of morning stiffness in arthralgia and early arthritis: results from the Groningen EARC, Leiden EARC, ESPOIR, Leiden EAC and REACH.” Arthritis Res Ther 17: 108.

van Nies, J. A., E. Brouwer, D. P. de Rooy, F. A. van Gaalen, T. W. Huizinga, M. D. Posthumus and A. H. van der Helm-van Mil (2013). “Reasons for medical help-seeking behaviour of patients with recent-onset arthralgia.” Ann Rheum Dis 72(8): 1302-1307.

van Nies, J. A., A. Krabben, J. W. Schoones, T. W. Huizinga, M. Kloppenburg and A. H. van der Helm-van Mil (2014). “What is the evidence for the presence of a therapeutic window of opportunity in rheumatoid arthritis? A systematic literature review.” Ann Rheum Dis 73(5): 861-870.

van Nies, J. A., H. W. van Steenbergen, A. Krabben, W. Stomp, T. W. Huizinga, M. Reijnierse and A. H. van der Helm-van Mil (2015). “Evaluating processes underlying the predictive value of baseline erosions for future radiological damage in early rheumatoid arthritis.” Ann Rheum Dis 74(5): 883-889.

van Steenbergen, H. W., D. Aletaha, L. J. Beaart-van de Voorde, E. Brouwer, C. Codreanu, B. Combe, J. E. Fonseca, M. L. Hetland, F. Humby, T. K. Kvien, K. Niedermann, L. Nuno, S. Oliver, S. Rantapaa-Dahlqvist, K. Raza, D. van Schaardenburg, G. Schett, L. De Smet, G. Szucs, J. Vencovsky, P. Wiland, M. de Wit, R. L. Landewe and A. H. van der Helm-van Mil (2017). “EULAR definition of arthralgia suspicious for progression to rheumatoid arthritis.” Ann Rheum Dis 76(3): 491-496.

van Steenbergen, H. W., L. Mangnus, M. Reijnierse, T. W. Huizinga and A. H. van der Helm-van Mil (2016). “Clinical factors, anticitrullinated peptide antibodies and MRI-detected subclinical inflammation in relation to progression from clinically suspect arthralgia to arthritis.” Ann Rheum Dis 75(10): 1824-1830.

Verheul, M. K., S. Bohringer, M. A. M. van Delft, J. D. Jones, W. F. C. Rigby, R. W. Gan, V. M. Holers, J. D. Edison, K. D. Deane, K. M. J. Janssen, J. Westra, M. Brink, S. Rantapaa-Dahlqvist, T. W. J. Huizinga, A. H. M. van der Helm-van Mil, D. van der Woude, R. E. M. Toes and L. A. Trouw (2018). “Triple Positivity for Anti-Citrullinated Protein Autoantibodies, Rheumatoid Factor, and Anti-Carbamylated Protein Antibodies Conferring High Specificity for Rheumatoid Arthritis: Implications for Very Early Identification of At-Risk Individuals.” Arthritis Rheumatol 70(11): 1721-1731.

Willemze, A., M. P. van der Linden, S. le Cessie, L. A. Trouw, R. E. Toes, T. W. Huizinga and A. H. van der Helm-van Mil (2011). “The window of opportunity in ACPA-positive rheumatoid arthritis is not explained by ACPA characteristics.” Ann Rheum Dis 70(9): 1697-1698.

Young, K. A., K. D. Deane, L. A. Derber, J. M. Hughes-Austin, C. A. Wagner, J. Sokolove, M. H. Weisman, J. H. Buckner, T. R. Mikuls, J. R. O'Dell, R. M. Keating, P. K. Gregersen, W. H. Robinson, V. M. Holers and J. M. Norris (2013). “Relatives without rheumatoid arthritis show reactivity to anti-citrullinated protein/peptide antibodies that are associated with arthritis-related traits: studies of the etiology of rheumatoid arthritis.” Arthritis Rheum 65(8): 1995-2004.

Zaccardelli, A., H. M. Friedlander, J. A. Ford and J. A. Sparks (2019). “Potential of Lifestyle Changes for Reducing the Risk of Developing Rheumatoid Arthritis: Is an Ounce of Prevention Worth a Pound of Cure?” Clinical Therapeutics 41(7): 1323-1345.

Zack, D. J., M. Stempniak, A. L. Wong and R. H. Weisbart (1995). “Localization of an Fc-binding reactivity to the constant region of human IgG4. Implications for the pathogenesis of rheumatoid arthritis.” J Immunol 155(10): 5057-5063.

We claim:

1. A method of stabilizing a blood sample for serologic analysis comprising:
obtaining a blood sample wherein the blood sample includes at least one biomarker; and
suspending the blood sample in a blood stabilizing solution;
wherein an amount of the solution is sufficient to preserve the biomarker;
wherein the solution consists of tris-HCL in buffered saline, bovine serum albumin, tyrosine, calcium chloride, trehalose, a preservative, and water;
wherein a concentration of the bovine serum albumin is at least 0.5%;
wherein a concentration of the tyrosine is at least 0.04%;
wherein a concentration of the calcium chloride is at least 0.05%; and
wherein a concentration of the trehalose is at least 1.0%.

2. The method of claim 1, wherein a concentration of the tris-HCL in buffered saline is at least 0.1M.

3. The method of claim 1, wherein a concentration of the preservative is at least 0.001%.

4. The method of claim 1, wherein the preservative is 2-methyl-4-isothiazolin-3-one solution.

5. The method of claim 1, wherein the biomarker is preserved for at least 7 days.

6. The method of claim 1, wherein the biomarker identifies an autoimmune disease.

7. The method of claim 1, wherein the biomarker is an immunoglobulin.

8. The method of claim 1, wherein the biomarker is rheumatoid factor IgM, rheumatoid factor IgA, or an anti-cyclic citrullinated peptide.

9. A method of screening an individual for an autoimmune disease comprising analyzing a blood sample from an individual wherein the blood sample is stabilized according to the method of claim 8, and wherein the autoimmune disease is rheumatoid arthritis, celiac disease, systemic lupus erythematosus, or Sjogren's syndrome.

10. A method of mass screening individuals for rheumatoid arthritis comprising:
screening a general population;
identifying an individual with a high risk factor for developing rheumatoid arthritis;
collecting a blood sample from the individual wherein the blood sample includes at least one biomarker;
suspending the blood sample in a stabilizing solution,
wherein an amount of the solution is sufficient to preserve the biomarker;
wherein the solution consists of tris buffered saline, bovine serum albumin, tyrosine, calcium chloride, trehalose, a preservative, and water;
wherein a concentration of the bovine serum albumin is at least 0.5%;
wherein a concentration of the tyrosine is at least 0.04%;
wherein a concentration of the calcium chloride is at least 0.05%; and
wherein a concentration of the trehalose is at least 1.0%;
analyzing the blood sample to determine a biomarker type and a biomarker level; and
determining if the individual is at high risk for developing rheumatoid arthritis based on the biomarker type and the biomarker level.

11. The method of claim 10, wherein the blood sample is collected via a finger prick.

12. The method of claim 11, wherein the blood sample is at least 20 μL of whole blood.

13. The method of claim 10, wherein the biomarker is rheumatoid factor IgM, rheumatoid factor IgA, or an anti-cyclic citrullinated peptide.

14. The method of claim 10, wherein a concentration of the tris buffered saline is about 0.1M.

15. The method of claim 10, wherein a concentration of the preservative solution is at least 0.001%.

16. The method of claim 10, wherein the biomarker is preserved for at least 7 days.

17. The method of claim 10, wherein the biomarker type and the biomarker level is determined by an ELISA constructed to identify rheumatoid factor IgM, rheumatoid factor IgA, and anti-cyclic citrullinated peptide.

18. The method of claim 17, wherein a biomarker level of rheumatoid factor IgM and rheumatoid factor IgA and anti-cyclic citrullinated peptide higher than about 95% of a normal population indicates a high risk of developing rheumatoid arthritis.

19. The method of claim 17, wherein a biomarker level of anti-cyclic citrullinated peptide higher than about 95% of a normal population indicates a high risk of developing rheumatoid arthritis.

20. A kit for mass rheumatoid arthritis screening comprising:
a device to obtain a blood sample from an individual; and
a blood sample collection vial including a label and a blood stabilizing solution;
wherein an amount of the solution is sufficient to preserve a blood sample biomarker;
wherein the solution consists of tris buffered saline, bovine serum albumin, tyrosine, calcium chloride, trehalose, a preservative solution, and water;
wherein a concentration of the bovine serum albumin is at least 0.5%;
wherein a concentration of the tyrosine is at least 0.04%;
wherein a concentration of the calcium chloride is at least 0.05%;
wherein a concentration of the trehalose is at least 1.0%; and
wherein the biomarker is rheumatoid factor IgM, IgA, or an anti-cyclic citrullinated peptide.

21. A kit for screening an individual for an autoimmune disease comprising:
a device to obtain a blood sample from an individual; and
a blood sample collection vial including a label and a blood stabilizing solution;
wherein an amount of the solution is sufficient to preserve a blood sample biomarker;
wherein the solution consists of tris buffered saline, bovine serum albumin, tyrosine, calcium chloride, trehalose, a preservative solution, and water;
wherein a concentration of the bovine serum albumin is at least 0.5%;
wherein a concentration of the tyrosine is at least 0.04%;
wherein a concentration of the calcium chloride is at least 0.05%;
wherein a concentration of the trehalose is at least 1.0%;
wherein the biomarker identifies an autoimmune disease; and
wherein the autoimmune disease is rheumatoid arthritis, celiac disease, systemic lupus erythematosus, or Sjogren's syndrome.

22. A non-transitory machine-readable medium storing instructions that, when executed by one or more processors, cause the one or more processors to perform steps comprising:
screening a general population;
identifying an individual with a high risk factor for developing rheumatoid arthritis;
analyzing a blood sample collected from the individual;
wherein the blood sample includes at least one biomarker to determine a biomarker type and a biomarker level; and
determining if the individual is at high risk for developing rheumatoid arthritis based on the biomarker type and the level.
